(19) United States
(12) Patent Application Publication
(10) Pub. No.: US 2006/0058658 A1
(43) Pub. Date: Mar. 16, 2006

(54) Communications between co-located operating systems for medical diagnostic ultrasound and other systems

(75) Inventors: Ricky L. King, Sunnyvale, CA (US); Kenneth M. Wright, San Jose, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 10/940,236

(22) Filed: Sep. 13, 2004

(51) Int. Cl. A61B 8/00

(52) U.S. Cl. 600/437

(57) ABSTRACT

Two different operating systems, such as one using Windows real-time extensions and another using Windows NT or other non-real-time operating system, may be implemented on a same system or hardware, such as being co-located on a same processor, without changing client software. Two or more software processes are run on a same processor or system. The software or operating systems communicate using a socket application programming interface. The use of socket communications allows the processes to communicate as if the processes were on different systems. Socket communications between the two operating systems are intercepted by the layered service provider before being provided to the hardware for external transport. The socket communications are re-routed to the socket stack of the destination operating system. The socket communication using sockets is transparent to the application or middleware layer software. By providing for socket communications between two co-located operating systems, the implementation cost and development risk caused by using a shared memory may be avoided.

### BACKGROUND

The present invention relates to embedded systems with multiple operating systems or co-located processes. In particular, communications between co-located operating systems or processes are provided for medical diagnostic ultrasound or other systems.

Many complex systems, such as medical imaging systems, include different software. Some software is run on one type of operating system, such as Microsoft Windows NT. Other software is run on a real-time operating system. Different sets of hardware, such as two different motherboards, are provided for the different software. A socket based protocol may be used to communicate between the two sets of hardware. Network interface cards, Ethernet cabling, routers or other networking equipment provides a socket based communication mechanism. The software using the socket based communication may be designed for the given sets of hardware, limiting the ability to change to alternative communications.

The different processes are alternatively implemented on a same system or processor. For example, Windows real-time extensions allow a real-time operating system to run in a co-located manner with the Windows NT Operating System. To communicate between the two processes or associated operating systems, mailbox or shared memory communications are provided. Both operating systems manage a shared file system with persistent information. Overhead communications are used to manage the shared memory so that one process may store data to the shared memory for retrieval by the other process. Use of shared memory may introduce delay as well as requiring overhead processing for communicating through the shared memory.

### BRIEF SUMMARY

By way of introduction, preferred embodiments described below include methods for operating a computer system, computer systems, and computer readable storage medium having instructions for operating computer systems. Two different operating systems, such as one using Windows real-time extensions and another using Windows NT or other non-real-time operating system, may be implemented on a same system or hardware, such as being co-located on a same processor, without changing client software. Two or more software processes are run on a same processor or system. Software processes communicate using a socket application programming interface. The use of socket communications allows the processes to communicate as if the processes were on different systems. Socket communications between the two operating systems are intercepted by the layered service provider before being provided to the hardware for external transport. The socket communications are re-routed to the socket stack of the destination operating system. The socket communication is transparent to the application or middleware layer software. By providing for socket communications between two co-located operating systems the cost of implementing a new communication protocol may be avoided.

In a first aspect, a method is provided for operating a medical diagnostic ultrasound imaging system. Operations of the medical diagnostic ultrasound imaging system are controlled in real-time with a real-time operating system. Operations of the medical diagnostic imaging system are also controlled with a non-real-time operating system. The two operating systems are operable to share hardware of the medical diagnostic ultrasound imaging system. The real-time operating system communicates with the non-real-time operating system with the socket communications.

In a second aspect, a method is provided for operating a computer system. The computer system is controlled in real-time with a real-time operating system and also controlled with a non-real-time operating system. The two operating systems are co-located. Socket communications are used to communicate between the two operating systems.

In a third aspect, a computer readable storage media is provided. The computer readable storage medium has stored therein data representing instructions executable by a computer for operating a medical diagnostic imaging system. The instructions are for controlling operations of the medical diagnostic imaging system in real-time with a real-time operating system and with a non-real-time operating system. The two operating systems are operable to share hardware of the medical diagnostic imaging system. The two operating systems communicate with each other using socket communications.

In as fourth aspect, an operating system is provided for a computer. A processor is operable to run an operating system with real-time extensions and a non-real-time operating system substantially simultaneously. A port is operable to provide socket communications external to the processor. The two operating systems are operable to communicate with socket communications intercepted prior to delivery to the port.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different view.

FIG. 1 is a block diagram of one embodiment of a computer system using different application processes;

FIG. 2 is a graphical representation of a software stack of one embodiment for providing socket communications between different applications; and

FIG. 3 is a flow chart diagram of one embodiment of a method for communicating between different control processes.

### DETAILED DESCRIPTION OF THE DRAWINGS AND PRESENTLY PREFERRED EMBODIMENTS

Application software implemented on two different operating systems, such as real-time and non-real-time operating systems, of a same processor or system communicate using socket calls of a service provider interface. The communications between applications run on same processor are transparent to the applications, avoiding changes in the application or client software for communicating between co-located processes. Implementation cost and development risk is decreased by avoiding shared memory access or avoiding external routing of communications between the processes. Microsoft service provider interface or other similar protocol stack provides the transparent socket communications between real-time and non-real-time components of an imbedded system.

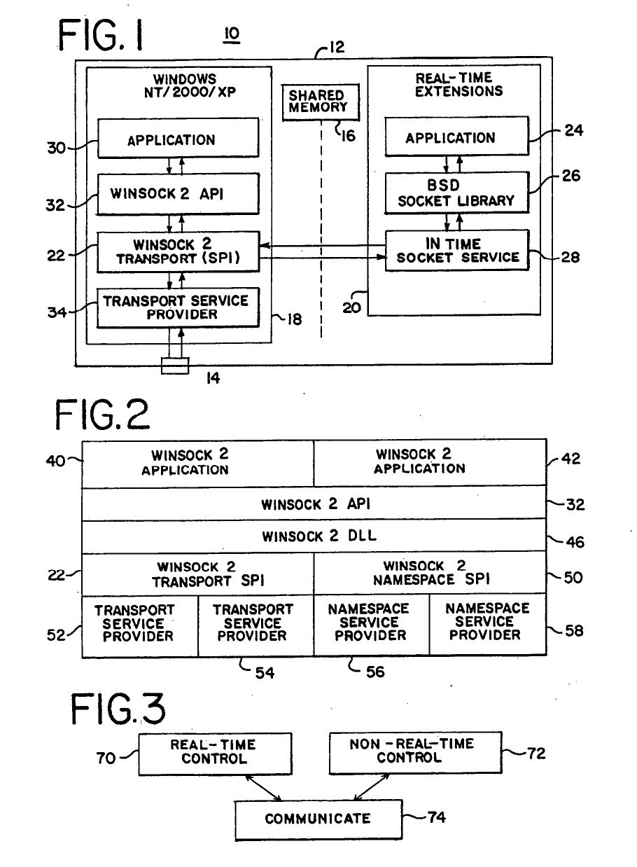

FIG. 1 shows one embodiment of a system 10 for a computer, medical imaging system, medical diagnostic ultrasound imaging system or other systems. The system 10 includes a processor 12, a port 14 and a shared memory 16. Additional, different or fewer components may be provided, such as the processor 12 being implemented on a motherboard, or other circuit board. As another example, a plurality of ports 14 is provided.

The processor 12 is a general processor, digital signal processor, control processor, circuit board, computer, microprocessor, combinations thereof or other now known or later developed device for running software or implementing software processes. The processor 12 is operable to run two or more different operating systems 18, 20. The operating systems share the same hardware, but may be configured to control different aspects of the system 10. For example, non-real-time and real-time operating systems are provided. The real-time operating system runs with real-time extensions, such as provided by a Windows operating system. The non-real-time operating system is a general application personal computer operating system or other system with a service provider interface 22 operable to control transfer of data on the port 14. In one embodiment, the general application personal computer operating system is a Windows NT, 2000 or XP system. Other Windows or non-Windows based general application operating systems may be provided, such as Linux based operating systems. In alternative embodiments, two real-time or two non-real-time operating systems are used.

The operating systems are implemented by the processor 12 at substantially a same time, such as using shared processing of the same processor. Substantially simultaneous operation includes infrequent operations by one operating system relative to the other operating system where both are resident or running on the processor 12 without substantial transfers of information to or from memory associated with loading an operating system or awaking an operating system. For example, a real-time operating system 20 owns, controls or otherwise manages the processor 12 with the non-real-time operating system 18 being a low priority thread of the real-time operating system.

The real-time operating system 20 includes a library or collection of services and facilities for using the processor 12, the system 10, a shared memory 16, drives or other components. In one embodiment, the operating system 20 is a Windows real-time extensions operating system. The operating system 20 includes application processes 24, a BSD socket library 26 and an IN-time socket service 28. Additional, different or fewer software components may be provided.

The application 24 performs operations for controlling portions of the hardware of the system 20 partitioned to the real-time operating system 20. For example, in a medical diagnostic imaging system, the real-time operating system 20 and associated applications 24 are operable to control imaging as a function of imaging parameters. The applications 24 control the various functions along an imaging path. For ultrasound systems, parameters may control operation of a transmit beamformer, receive beamformer, filter, detector, scan converter or other imaging characteristics. By using real-time extensions and operating in real-time, guaranteed processing or control for imaging is provided so that real-time imaging may result. The operating system 20 and the associated applications 24 respond to interrupts within a guaranteed or set amount of time, such as timing processing of interrupts on priorities.

The socket library 26 and socket service 28 are two examples of software packages or protocol stacks for performing socket calls. In alternative embodiments, different now known or later developed software for socket processing may be provided. The in-time socket service 28 provides socket communications using real-time extensions. The socket library 26 provides a library of calls or associated socket communications available. The socket library 26 is a library of TCP/IP information for communicating between operating systems, such as operating systems associated with different processors. Socket communications may include date to be transferred with software that is transparent to the requesting application.

The non-real-time operating system 18 includes a collection or library of services and facilities for using the processor 12, the system 10, drives, the shared memory 16 or other components. The non-real-time operating system 18 includes application software 30, an application program interface 32, the service provider interface 22 and a transport service provider 34. Additional, different or fewer components may be provided. In one embodiment, the non-real-time operating system is implemented using Windows NT, 2000 or XP. Other now known or later developed non-real-time operating systems may be used. Non-real-time operation is provided using a round robin thread. Interrupts are processed as received or without priority. When a real-time operating system 20 implements the non-real-time operating system 18 as a low priority thread, the interrupts for the non-real-time operating system 20 are performed or scheduled if no real-time interrupts are currently scheduled.

The applications 30 control operations of hardware of the system 10 partitioned to the non-real-time operating system 18. For example, the applications 30 control a database of imaging parameters stored in the shared memory 16 or a remote random access memory. The applications 30 also control user interface components, such as keyboards, user input devices, displays, monitors or user output devices. The applications 30 may additionally or alternatively control graphics or other processes for display. Other distribution of control functions may be used.

The port 14 is a network interface card, Ethernet cable, router or other network equipment for communicating from the processor 12 to other processors or remote equipment. As shown in FIG. 1, the port 14 is managed by the transport service provider 34 of the non-real-time operating system 18. In alternative embodiments, the port 14 is managed by the real-time operating system 20 or by both operating systems 18, 20. The port 14 is operable for socket communications external to the processor 12. Both the real-time operating system 20 and the non-real-time operating system 18 are operable to communicate with remote devices through the port 14 using socket communications. Socket calls generated by the real-time operating system 20 and associated application 24 are routed by the socket service 28 to the service provider interface 22 of the non-real-time operating system 18. The socket library 26 and socket service 28 may also indicate an address for a given socket communications, such as an address for a device external to the processor 12. The service provider interface 22 implements a ubiquitous library of socket calls indicating the existence and location of a device for communications through the port 14. The service provider interface 22 implements sockets to move data through the port 14, such as by controlling the read operations through the port 14. The transport service provider 34 controls the hardware or drives the port 14 pursuant to instructions from the service provider interface 22. Socket communications received at the port 14 for the processor 12 are then routed to the appropriate application 20, 24 using the service provider interface 22.

FIG. 2 shows one embodiment of a software protocol stack implemented by the non-real-time operating system 18 and/or the real-time operating system 20. The protocol stack will be described with respect to the non-real-time operating system 18 as represented in FIG. 1. The protocol stack corresponds to a Winsock 2 software application program interface between the operating system 18 and TCP/IP protocol software associated with the port 14. In alternative embodiments, other interface software than Winsock 2 may be used, such as other now known or later developed software including the BSD socket library 26 and/or the IN-time socket service 28. The protocol stack is a network programming interface at the transport level in the ISO reference model. The stack includes two applications 40 and 42 for communicating between each other. The applications comprise middleware, intermediate level software or higher level software. The applications 40 and 42 are implemented on the two different operating systems 18 and 20 or between the processor 12 and a remote processor.

The applications 40, 42 communicate with the Winsock application program interface 32. The program interface provides a library of socket calls to act as a transport layer between the transmission control protocol (TCP) or user datagram protocol (UDP) and the applications 40 and 42 on the system. Software calls and routines may be referenced by the applications 40, 42 to access underlying network services using the application programming interface 32.

The dynamic link library 46 allows executable code modules to be loaded on demand and linked at run-time. Since the links are performed at run-time, a code may be altered or changed transparent to the applications 40 and 42.

The transport and name space service provider interfaces 22 and 50 define how a network or external communications through the port 14 interfaces with the operating system through the applications programming interface 32. This service provider interface 22, 50 allows the development of transport providers or protocol stacks as services. For example, the real-time socket established by the socket service 28 shown in FIG. 1 is an always running background service. The service provider interface 22, 50 supplies functions to set up connections, transfer data, exercise flow control, error control and other communications for transport and name space services. The name space service provider interface 50 identifies addresses and other network addresses and port numbers for services being used for communications with remote devices, such as through the Internet or other local network connection. The service provider interfaces a software layer directly above the hardware for providing standardized interfaces to manipulate PCMCIA cards, adapters and other sockets.

The transport service provider interface 22 includes a layered service provider's software which implements higher level custom communication functions. The software may be reused or rests on top of the sockets. The underlying base providers then implement the hardware data exchange with a remote endpoint as represented by the transport and name space service providers 52, 54, 56 and 58. The service providers 52, 54, 56, 58 are generally represented in FIG. 1 as the transport service provider labeled 34. The service providers implement the socket through the port 14. In response to a socket call, communications through the port 14 are generated by the transport service provider 34. Middleware communications software embedded in the application, such as applications 40, 42 manages the name space. The software of the stacks shown in FIG. 2 handles the synchronization with the socket, type of communication being implemented, such as a broadcast, acknowledgement, or a callback to process a message. The name space for matching a name with an IP address in port 14 is also implemented.

The software represented in FIG. 2 may be used to communicate between operating systems 18, 20 and associated applications 30, 24 co-located on the same processor 12 without use of the port 14 or a socket. The operating systems 18, 20 communicate with socket communications intercepted prior to delivery to the port 14. The service provider interface is used to provide transparent socket communications. A layered service provider is written to intercept socket calls before they are passed down to the hardware level. The protocol stack shown in FIG. 2 for co-located operating systems can use a layered service provider to intercept the socket calls. Socket calls for the different operating systems 18, 20 are associated with different addresses. The operating system with real-time extensions 20 acts as a client and the non-real-time operating system 18 acts as a server for accessing the port 14. Both operating systems 18, 20 are associated with a hardwired address or ports for intercommunications. In alternative embodiments, the real-time operating system 20 acts as a server to the non-real-time operating system 18 client. The service provider interfaces are implemented as drivers running in the background, allowing application and control software running on the same processor 12 but with different operating systems 18, 20 to communicate via socket calls or communications without having to change client software. The communications are handled through drivers rather than having to write data to the shared memory 16 by one operating system 18, 20 and reading the data out by the other operating system 20, 18.

The layered service provider of the service provider interface 22 on the non-real-time operating system 18 is operable to intercept socket communications associated with either the non-real-time operating system 18 or the operating system 20 using real-time extensions. The layered service provider is then operable to route the socket communications to a socket stream of the other one of the operating systems 20, 18. For example, socket calls addressed to the socket service 28 of the real-time operating system 20 by the non-real time operating system 18 are intercepted and sent to the socket service 28 within the same processor 12 without using the external communications of the port 14. Other calls from the application 30 for other hardware are passed on to the hardware or through the socket of the port 14. Calls originating from the real-time operating system 20 and associated application 24 are (1) sent to itself in a loop-back address or a local non-routing address or (2) communicated through a co-located Windows IP address. If addressed for the non-real-time operating system 18, the socket call generated by the real-time operating system 20 is intercepted by the layered service provider of the service provider interface 22 of the non-real-time operating system 18.

In an implementation for medical imaging, the non-real-time operating system and associated applications 30 manage a database of parameters for imaging characteristics and manage the user interface, such as user input selection of a type of imaging. The real-time operating system 20 and associated application 24 control the various imaging system components, such as beamformers, based on parameters communicated through socket communications from the non-real-time operating system 18. The real-time operating system 20 may provide time information or other data associated with imaging or feedback from the controlled hardware for recording, maintenance or display by the applications 30 of the non-real-time operating system 18. The socket communications for medical imaging or other computer system operations are provided where one operating system 18, 20 interacts with or uses information from the other operating system 20, 18.

FIG. 3 shows one embodiment of a method for operating a computer system, such as a medical diagnostic imaging system or medical diagnostic ultrasound imaging system. The software for implementing operation of the systems is stored on a computer readable storage medium as instructions executable by the computer for operating the system. For example, the instructions are stored in a buffer, cache, RAM, ROM, removable media, hard drive, combinations thereof or other now known or later developed memory. Additional, different or fewer acts than shown in FIG. 3 may be provided.

In act 70, a computer system or other system is controlled in real time with the real time operating system. For example, operations of a medical diagnostic ultrasound imaging system are controlled in real time. Any of various operations may be controlled, such as operations associated with beamformers, detectors, scan converters, filters, displays, transmitters, receivers or other now known or later developed medical imaging hardware. Other operations include calculations, user interface options and/or communications operations. The operating system controls operations through one or more applications as part of, as thread within, resident on, run pursuant to or managed by the operating system.

Control of the operations is implemented in real time. For example, a guaranteed response time to an external interrupt is provided by the operating system. By using real-time extensions or other processes, different operations or interrupts may be controlled based on assigned priority. Non-real time operations may be provided as part of the control of operations with a real-time operating system. For example, the real-time operating system implements the non-real-time operating system in a low priority thread. Different interrupts are owned by the two different operating systems, such as associated with the hardware partitioning between the operating systems.

In act 72, the computer system is controlled with the non-real time operating system. For example, operations of the medical diagnostic ultrasound imaging system are controlled with the non-real time operating system. In one embodiment, the non-real time operating system is a general application personal computer operating system, such as a windows based system. The operations controlled include any of the operations of the computer system or medical imaging system assigned to the particular operating system. For example, user interface and/or external communications operations are controlled with the applications running on the non-real time operating system. Input and/or output user interface operations may be controlled. Non-real time operation is performed using a round robin queuing technique of interrupts. Other non-real time processes may be used, such as assigning a priority or order for performing interrupts that is free of reorganization as a function of time.

The non-real time operating system and the real time operating system of act 70 are collocated, such as being run on a same processor. For example, the real time operating system manages the non-real time operating system as an application or thread or vice versa. The operating systems are operable to share hardware of the computer or medical imaging system. For example, the memory and/or processor are shared. Other hardware within the system is partitioned between the different operating systems, such as partitioning remote devices within the system. For example, the non-real time operating system runs applications or drivers for control of user input devices, displays, network cards, external communications ports, and/or other hardware of a personal computer system. Hardware for embedded systems that operate in real time, such as imaging systems, is partitioned to applications of the real-time operating system.

In act 74, communications between the two operating systems are performed with socket communications. The communication is performed with a service provider interface. For example, a socket call from the non-real time operating system is received by a service provider interface of the general application personal computer operating system, such as associated with the non-real time operating system. The hardware address is committed to a non-routing IP address of the real time operating system, such as associated with the real time BSD socket service. The socket call from the non-real time operating system is duplicated within the layered service provider rather than routing to a hardware address. One set of the duplicated socket calls have semantics for the non-real time operating system and the other set has semantics for the real time operating system. The duplicating intercepts the socket call without passing to the hardware transport. The layered service provider of the protocol stack of the non-real time operating system intercepts the call, imports the socket call to the desired operating system, such as the non-real time or real time operating systems.

Socket calls from the real time service provider not indicated as a local address to the real time service provider are sent to the duplicated IP socket address. As a result, the socket calls are routed to the layered service provider of the non-real time operating system for duplication and insertion into the socket call stream of the non-real time operating system. Where the socket address indicates the non-real time operating system, the socket call is handled with software without passing to the hardware for communications externally. Where the IP socket address is associated with external communications, the layer service provider routes the socket call to the hardware for external communications. Similarly, where the non-real time operating system generates a socket communications address for the real time operating system, such an address associated with the BSD socket library, the call is intercepted before being sent to hardware for routing to the dedicated IP address of the real time operating system.

Since many different computer systems or embedded systems use socket communications for external or network related communications, the service provider may be altered to provide for socket communications of co-located processes. Embedded systems with both real time and non-real time operating systems may use the socket communications to reduce hardware calls and complexities while avoiding the need to change or alter application or client software.

As an alternative to intercepting socket communications at the service provider interface level, higher level software may be used for intercepting or initially routing the socket communications between operating systems, such as the API or DLL level. In yet other embodiments, both the non-real time and real time or only the real time operating system intercepts the socket calls, such as where the real time operating system or both systems control ports for external communications. Two non-real time or two real time operating systems may alternatively communicate using socket communications as discussed herein.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I claim:

1. A method for operating a medical diagnostic ultrasound imaging system, the method comprising:
(a) controlling first operations of the medical diagnostic ultrasound imaging system in real-time with a real-time operating system;
(b) controlling second operations of the medical diagnostic ultrasound imaging system with a non-real-time operating system, the real-time operating system and non-real-time operating systems operable to share hardware of the medical diagnostic ultrasound imaging system; and
(c) communicating between the real-time operating system and the non-real-time operating system with socket communications.

2. The method of claim 1 wherein (a) comprises guaranteeing response time to an external interrupt.

3. The method of claim 1 wherein (a) comprises controlling the first operations based on priority.

4. The method of claim 1 wherein (b) comprises controlling the second operations with round-robin scheduling.

5. The method of claim 1 wherein (a) and (b) comprise the real-time operating system managing the non-real-time operating system as a low priority thread.

6. The method of claim 1 wherein (a) comprises controlling medical imaging and wherein (b) comprises running a user interface with a general application personal computer operating system.

7. The method of claim 1 wherein (c) comprises intercepting socket calls without passing to hardware transport.

8. The method of claim 7 wherein (c) comprises intercepting the socket calls with a layered service provider.

9. The method of claim 7 wherein (c) comprises intercepting the socket calls with a protocol stack of the non-real-time operating system.

10. The method of claim 1 wherein (c) comprises communicating with a service provider interface.

11. The method of claim 1 wherein (c) comprises routing a call from the real-time operating system to a set port.

12. The method of claim 1 wherein (c) comprises porting a socket call of one of the real-time and non-real-time operating systems to a socket stream of the other of the real-time and non-real-time operating systems.

13. The method of claim 1 wherein (b) comprises controlling transport with a general application personal computer operating system, and wherein (c) comprises communicating with a service provider interface of the general application personal computer operating system.

14. A method for operating a computer system, the method comprising:
(a) controlling the computer system with a first operating system;
(b) controlling the computer system with second operating system different than the first operating system, the first operating system and second operating systems being co-located; and
(c) communicating between the first operating system and the second operating system with socket communications.

15. The method of claim 14 wherein (a) comprises controlling with real-time extensions.

16. The method of claim 14 wherein (b) comprises controlling the second operations with round-robin scheduling.

17. The method of claim 14 wherein (a) and (b) comprise the first operating system managing the second operating system as a low priority thread.

18. The method of claim 14 wherein (c) comprises intercepting the socket calls with a layered service provider.

19. The method of claim 18 wherein (c) comprises intercepting the socket calls with a protocol stack of the second operating system.

20. The method of claim 14 wherein (c) comprises communicating with a service provider interface.

21. The method of claim 14 wherein (c) comprises porting a socket call of one of the first and second operating systems to a socket stream of the other of the second and first operating systems.

22. A computer readable storage medium having stored therein data representing instructions executable by a computer for operating a medical diagnostic imaging system, the storage medium including instructions for:
(a) controlling first operations of the medical diagnostic imaging system in real-time with a real-time operating system;
(b) controlling second operations of the medical diagnostic imaging system with a non-real-time operating system, the real-time operating system and non-real-time operating systems operable to share hardware of the medical diagnostic imaging system; and
(c) communicating between the real-time operating system and the non-real-time operating system with socket communications.

23. The storage medium instructions of claim 22 wherein (a) comprises controlling the first operations based on priority and wherein (b) comprises controlling the second operations with round-robin scheduling.

24. The storage medium instructions of claim 22 wherein (a) and (b) comprise the real-time operating system managing the non-real-time operating system as a low priority thread.

25. The storage medium instructions of claim 22 wherein (a) comprises controlling medical imaging and wherein (b) comprises running a user interface with a general application personal computer operating system.

26. The storage medium instructions of claim 22 wherein (c) comprises intercepting the socket calls with a layered service provider.

27. The storage medium instructions of claim 26 wherein (c) comprises intercepting the socket calls with a protocol stack of the non-real-time operating system.

28. The storage medium instructions of claim 22 wherein (c) comprises communicating with a service provider interface.

29. The storage medium instructions of claim 22 wherein (c) comprises routing a call from the real-time operating system to a set port.

30. The storage medium instructions of claim 22 wherein (c) comprises porting a socket call of one of the real-time and non-real-time operating systems to a socket stream of the other of the real-time and non-real-time operating systems.

31. The storage medium instructions of claim 22 wherein (b) comprises controlling transport with a general application personal computer operating system, and wherein (c) comprises communicating with a service provider interface of the general application personal computer operating system.

32. An operating system for a computer, the operating system comprising:
a processor operable to run an operating system with real-time extensions and a non-real-time operating system substantially simultaneously; and
a port operable to provide socket communications external to the processor;
wherein the real-time operating system and the non-real-time operating system are operable to communication with socket communications intercepted prior to delivery to the port.

33. The operating system of claim 32 wherein the non-real-time operating system comprises a general application personal computer operating system with a service provider interface operable to control transfer of data on the port.

34. The operating system of claim 32 wherein a layered service provider of the non-real-time operating system is operable to intercept the socket communication of one of the non-real-time operating system and the operating system with the real-time extensions and operable to route the socket communication to a socket stream of the other one of the non-real-time operating system and operating system with the real-time extensions.

35. The operating system of claim 32 wherein the processor comprises a processor of a medical diagnostic imaging system, the non-real-time operating system is operable to control a database of imaging parameters and the operating system with the real-time extensions is operable to control imaging using the imaging parameters.
